(19) United States
(12) Patent Application Publication
(10) Pub. No.: US 2017/0267993 A1
(43) Pub. Date: Sep. 21, 2017

(54) DIRECTED EVOLUTION USING TURBIDOSTAT FOR INCREASED SPECIFIC GROWTH RATE AND REDUCED LIGHT-HARVESTING ANTENNA SIZE OF PHOTOSYNTHETIC MICROORGANISMS FOR INCREASED PHOTOSYNTHETIC EFFICIENCY

(71) Applicant: ExxonMobil Research and Engineering Company, Annandale, NJ (US)

(72) Inventors: Joseph C. Weissman, High Bridge, NJ (US); Kelsey M. McNeely, Lambertville, NJ (US)

(21) Appl. No.: 15/454,003

(22) Filed: Mar. 9, 2017

Related U.S. Application Data
(60) Provisional application No. 62/309,616, filed on Mar. 17, 2016.

(51) Int. Cl. C12N 15/01; C12R 1/89

(52) U.S. Cl. CPC C12N 15/01; C12R 1/89

(57) ABSTRACT

Methods are provided to select strains of photosynthetic microorganisms for enhanced photosynthetic efficiency or biomass accumulation. Strains are mutagenized, and then grown under high light in a turbidostat. Microorganisms created by this process are also described, as well as methods of using such isolated microorganisms for biomass production.

### CROSS-REFERENCE TO RELATED APPLICATIONS

This application claims priority to U.S. Provisional Application Ser. No. 62/309,616 filed on Mar. 17, 2016, herein incorporated by reference in its entirety.

### FIELD

This disclosure relates to methods of selecting photosynthetic microbial strains/mutants having desirable properties, as well as the strains/mutants themselves (e.g., in cultures) and methods of using them (e.g., to produce biomass).

### BACKGROUND

Directed evolution is a powerful technique for improving the utility of a biological system for a given industrial application. For example, Pourmir & Johannes (2012) Comp. & Struct. Biotechnol. J 2(3):e201209012 report directed evolution techniques to improve xylanase activity in Chlamydomonas reinhardtii.

Nakajima & Ueda (1997) J. Appl. Phycol. 9:503-10 report mutagenizing Synechocystis and then screening for mutants with reduced pigment content. This is a strictly artificial selection process, and not a directed evolution process involving natural selection.

Mussgnug et al. (2007) Pl. Biotechnol. J. 5:802-14 report directed mutagenesis of light-harvesting antenna proteins to improve photosynthetic efficiency. This technique requires that one know ex ante which genes are relevant to the efficiency phenotype, and thus cannot be used to create as-yet unknown efficiency improvements.

U.S. Patent Application Publication No. 2014/0356902 reports methods for determining and/or engineering photosynthetic mutant algal strains by causing genetic mutations in a group of wild-type photosynthetic microorganisms to form genetic mutant strains and screening the genetic mutant strains for photosynthetic efficiency in mass cultures.

U.S. Patent Application Publication No. 2015/0087014 reports methods to select strains of algal cells for biomass accumulation, in which strains of algal cells co-cultured in a vessel are exposed to pre-specified illumination profiles under controlled conditions. Algal properties are measured and superior strains are designated for further cultivation and/or study.

### SUMMARY

Methods are described herein for selection of a strain of photosynthetic microorganisms for increased biomass accumulation in photosynthetic culture, based on specific growth rate. These methods comprise exposing at least two different strains of photosynthetic microbes to high light and/or low light for a period of time. In the methods described herein, the at least two different strains are co-cultured in a turbidostat, and at least one strain is a mutant strain. In certain embodiments, cells are mutagenized prior to the co-culturing, to generate a population of mutants that can be selected during these directed evolution processes.

Cells produced by these selection methods are also described herein. The photosynthetic microbes described herein show higher specific growth rates under high light conditions than are achieved with wild-type and prior-art engineered strains. As a result, the cells described herein are better able to accumulate biomass in a given unit of time than are corresponding wild-type or prior art strains.

Also described herein are methods of accumulating biomass by culturing the photosynthetic microbes described herein.

### DETAILED DESCRIPTION OF THE EMBODIMENTS

### Definitions

Unless defined otherwise, all technical and scientific terms used herein have the same meaning as commonly understood by one of ordinary skill in the art. Unless otherwise required by context, singular terms shall include pluralities and plural terms shall include the singular. For example, as used in the present disclosure and claims, singular forms “a,” “an,” and “the” include the plural unless context clearly dictates otherwise.

All publications, patents and other references mentioned herein are incorporated by reference in their entireties for all purposes as if each individual publication or patent application were specifically and individually indicated to be incorporated by reference. In case of conflict between definitions in the present text and those in the material incorporated by reference, the definitions in the present text will control over those incorporated by reference.

Although methods and materials similar or equivalent to those described herein can be used in practice or testing of the methods and microbes described herein, suitable methods and materials are described below. The materials, methods, and examples described herein are illustrative only and are not intended to be limiting. Other features and advantages of the methods and microbes described herein should be apparent from the detailed description and from the claims.

To facilitate an understanding of the present disclosure, a number of terms and phrases are defined below.

Wherever embodiments are described herein with the language “comprising,” otherwise analogous embodiments described in terms of “consisting of and/or “consisting essentially of are also provided.

The term “and/or” as used in a phrase such as “A and/or B” herein is intended to include “A and B”, “A or B”, “A”, and “B”.

The terms, “cells”, “cell cultures”, “cell line”, “recombinant host cells”, “recipient cells” and “host cells” as used herein, include the primary subject cells and any progeny thereof, without regard to the number of transfers. It should be understood that not all progeny are exactly identical to the parental cell (due to deliberate or inadvertent mutations or differences in environment); however, such altered progeny are included in these terms, so long as the progeny retain the same functionality as that of the originally transformed cell.

The terms “naturally-occurring” and “wild-type” (WT) refer to a form found in nature. For example, a naturally occurring or WT nucleic acid molecule, nucleotide sequence, or protein may be present in, and isolated from, a natural source, and is not intentionally modified by human manipulation. Similarly, a WT organism can be found in natural environment, and such an organism has not been genetically modified by human agency, nor has it descended from an ancestral organism that was genetically modified by human agency. In this context, “genetic modification” does not include cross-breeding and artificial selection techniques that do not involve direct enzymatic and chemical manipulation of nucleotide sequences.

The organism present at the start of a mutagenesis procedure is the “parental” organism. Although a wild-type organism can be a parental organism in a given mutagenesis operation, not all parental organisms are wild-type organisms.

A “turbidostat” is a culture vessel that is capable of monitoring optical density of a cell culture, and of diluting the culture as necessary to maintain a constant optical density despite cell growth within the culture.

As used herein, when a comparison is made between a given microorganism and a corresponding “control” microorganism, the “control” microorganism is substantially identical to the given microorganism, except for a single modification in question. For example, if a given microorganism has been selected in a directed evolution process on the basis of a mutation in the microorganism's pbcA gene, then the “control” microorganism is the otherwise unmodified descendant of the selectant's parent cell, which lacks the pbcA gene mutation. The “control” microorganism is substantially identical to the given microorganism to which a comparison is being made. In this context, “substantially” identical conveys that the control microorganism has not acquired any additional mutations that would materially affect the trait being compared between the control and given microorganisms. For example, if biomass accumulation is being compared between (1) a given microorganism with a pbcA gene mutation and (2) a corresponding control microorganism, the control microorganism cannot have suffered a frame-shift mutation in its own pbcB gene that results in a prematurely truncated PbcB enzyme. Such a mutation would be material, and therefore the control would not be substantially identical. However, the control microorganism could include an adventitious but silent mutation in its pbcB gene, because such a mutation would not have a material effect on PbcB enzymatic activity, and would therefore be insubstantial.

The term “photosynthetic organism” as used herein is any prokaryotic or eukaryotic organism that can perform photosynthesis. Photosynthetic organisms include higher plants (i.e., vascular plants), bryophytes, algae, and photosynthetic bacteria. The term “algae” includes, but is not limited to, a species of Bacillariophyceae (diatoms), Bolidomonas, Chlorophyceae (green algae), Chrysophyceae (golden algae), Carophyceae, Cyanophyceae (cyanobacteria), Eustigmatophyceae (pico-plankton), Glaucocystophytes, Pelagophytes, Phaeophyceae (brown algae), Prasinophyceae (pico-plankton), Raphidophytes, Rhodophyceae (red algae), Synurophyceae and Xanthophyceae (yellow-green algae). The term “algae” includes microalgae. The term “microalgae” as used herein refers to microscopic, single-celled algae species including, but not limited to, eukaryotic single-celled algae of the Bacillariophyceae, Chlorophyceae, Prasinophyceae, Trebouxiophyceae, and Eustigmatophyceae classes. The term “photosynthetic bacteria” includes, but is not limited to, cyanobacteria, green sulfur bacteria, purple sulfur bacteria, purple non-sulfur bacteria, and green non-sulfur bacteria.

The term “strain” as used herein refers to a genetic variant or subtype of a micro-organism (e.g., alga, bacterium, or protist). The phrase “at least two strains” can embrace two or more different species, two or more different subspecies within a single species, two or more different mutant members of the same species or the same subspecies, and/or any combination of these categories. The “dominant strain” is a strain, the cells of which constitute a plurality of the cells in a mixed culture; therefore, when three or more strains are cultured together, a single strain can be the “dominant strain” even without attaining a numerical majority of the cells within the culture. For example, if there are three cultures being co-cultured (A, B, & C) that begin in equal proportions (1:1:1), but after a number of rounds of selection, A=40% of the cells, B=30%, and C=30%, then A would the “dominant” strain, even though there are still more “non-A” cells in the culture than A cells. In other words, depending on the context, including the number of strains present in a mixed culture, a strain comprising 10%, 20%, 30%, 40% 50%, 60% 70%, 75%, 80%, 85%, 90%, 95%, 96%, 97%, 98%, 99%, or even 100% of the cells in the culture can be designated the “dominant strain.”

The term “biomass” as used herein, refers in general to organic matter produced by a biological cell. The renewable biological resource can include microbial materials (including algal materials), fungal materials, plant materials, animal materials, and/or materials produced biologically. “Biomass” should be understood to include proteins, lipids, and polysaccharides, whether retained within a biological cell or excreted from a biological cell, in addition to other molecules synthesized by a biological cell. As used herein, biomass is “excreted” from a cell whenever it is found outside of a cell, regardless of whether or not the egress of biomass occurred as a result of cellular activity.

The term “photosynthetic efficiency,” as used herein, refers to a measure of the ability of a photosynthetic organism to convert energy from actinic light into biomass. As photosynthetic efficiency increases, a larger percentage of the actinic light energy that a photosynthetic organism encounters is converted into biomass. In certain circumstances it is useful to speak of “relative photosynthetic efficiency.” In these circumstances, a particular photosynthetic organism is set as a reference standard. The photosynthetic efficiency of another photosynthetic organism is described in relation to the reference standard photosynthetic organism (e.g., 50% as efficient, 200% as efficient, etc.). A “photosynthetic efficiency characteristic” is a feature or element of a photosynthetic cell that affects or reflects the photosynthetic efficiency that a cell achieves under on or more circumstances. Exemplary photosynthetic efficiency characteristics include specific growth rate, pigment content (including chlorophyll content), oxygen evolution, carbon fixation, and tolerance of biomembranes (including chloroplastic membranes) to electrical potential.

“Photosaturating light conditions” are light conditions in which the cell is inundated with illumination, such that the amount of light energy entering the cell exceeds the cell's capacity to convert light energy into biomass. Therefore, photosaturating light conditions depress photosynthetic efficiency, because portions of energy entering the cell are simply lost for incapacity to process them. Photosaturating light conditions can induce the activation of a cells nonphotochemical quenching mechanisms, which result in light energy being dissipated as heat instead of being fixed into biomass. Photosaturating conditions that so greatly exceed the cell's capacities that the excess energy damages the cell's photosynthetic machinery are referred to as “photoinhibitory light conditions.” Photoinhibition may be measured using the decrease of Pₘₐₓ (the maximaun photosynthetic rate measured as oxygen evolution or radioactive carbon uptake) with time in high light. Nakajima & Ueda (1997), Mussgnug et al. (2007), and others have used traditional screening methods to develop antenna mutants, and have shown that these mutants were more resistant to photo inhibition than the parent strains. Han et al. (2000) J. Plankton Res. 22(5):865-85 report a model—based on reaction center damage and repair—which assumes the rate of photoinbition to be a function of the rate of photon arrival (photons harvested by antenna), such that the consequence for growth rate is a combination of the rate of photon arrival and the rate at which damaged reaction centers are repaired.

As used herein “pond” means any open body of water, whether naturally-occurring or man-made, including ponds, canals, trenches, lagoons, channels, or raceways. The open pond can have a depth of from about 3 cm to about 500 cm, and can typically have a depth of from about 5 cm to about 100 cm, such as from about 8 cm to about 50 cm, or from about 10 cm to about 40 cm.

The terms “artificial light” or “artificial source of light” are used herein to refer to any type of light other than sunlight. Thus, artificial light can include incandescent sources, fluorescent sources, light emitting diode sources, or any other convenient source for generating light. The opposite of “artificial” light is “natural” light.

When used in reference to a trait or characteristic (e.g., specific growth rate, rate of oxygen evolution, etc.), the words “diminished” and “attenuated” are interchangeable and mean “reduced in amount, degree, intensity, or strength.” Similarly, when used in reference to a trait or characteristic, the words “enhanced” or “increased” are interchangeable and mean “having a greater amount, intensity, degree, or strength.” In other words, “attenuated” and “diminished” are antonyms of “enhanced” and “increased.” An algal strain X is said to have an “enhanced” trait when the degree or intensity of the trait observed in strain X is greater than is observed in strain Y. The same applies, mutatis mutandis, to “increased,” “attenuated,” and “diminished.”

### Overview

Methods are described herein for directed evolution of photosynthetic microorganisms under selection conditions that select for higher specific growth rates under high light conditions. In these methods, a plurality of algae (i.e., two or more strains) are placed in a turbidostat and exposed to light based on a pre-specified illumination profile designed by the investigator. In certain embodiments, this illumination profile includes varying amounts of time at high or low light. For example, in certain embodiments, the plurality of photosynthetic microorganisms are exposed for at least 5 min. (e.g., at least 10 min., at least 20 min., at least 30 min., at least 45 min., at least 1 hour, at least 5 hours, at least 12 hours, at least 24 hours, at least 36 hours, at least 48 hours, at least 3 days, at least 4 days, at least 5 days, at least one week, at least 10 days, at least 12 days, or at least one fortnight) to high light. In certain embodiments, the plurality of photosynthetic microorganisms are exposed for at least 5 min. (e.g., at least 10 min., at least 20 min., at least 30 min., at least 45 min., at least 1 hour, at least 5 hours, at least 12 hours, at least 24 hours, at least 36 hours, at least 48 hours, at least 3 days, at least 4 days, at least 5 days, at least one week, at least 10 days, at least 12 days, or at least one fortnight) to low light. In certain embodiments, the selection under high light can be periodically interrupted with periods of low light, e.g., 10 hours of high light, then 1 hour of low light, followed by 10 more hours of high light, etc. By interrupting the high light with subsaturating light (e.g., 50 μEm⁻²s⁻¹) from time to time, one can select for mutants that do well in both high and low light (high intrinsic quantum efficiency). Additionally or alternatively, after selecting for increased specific growth rate in high light, one can culture at subsaturating light to select for improved specific growth rate at low light The duration of high and low light exposures can be adjusted as necessary to achieve a desired degree of selection intensity for a given trait.

It should be understood by one of skill in the art that the terms “high” and “low” are implicitly used in reference to a particular organism, and that a light condition that is “low” for one organism can be “high” for another. Depending on the organism, “high” light intensity is at least 150 μEm⁻²s⁻¹ (e.g., at least 200, at least 300, at least 500, at least 900, at least 1000, at least 1500, at least 2000, at least 2500, at least 3000, at least 3500, at least 4000, at least 4500, at least 5000, at least 5500, at least 6000, at least 7500 μEm⁻²s⁻¹, and in some cases ≧10000 μEm⁻²s⁻¹). Depending on the organism, “low” light intensity is not more than 200 μEm⁻²s⁻¹(e.g., not more than 150, not more than 100, not more than 75, not more that 50, not more than 25, not more than 15, not more than 10, not more than 5, or even less than 1 μEm⁻²s⁻¹). For instance, light intensities that are at or above the saturating light intensity for a particular organism can be thought of as “high” light intensities and/or light intensities that are below the saturating light intensity (particularly below 70% of the saturating light intensity, such as below 50% of the saturating light intensity, below 40% of the saturating light intensity, below 30% of the saturating light intensity, below 20% of the saturating light intensity, or below 10% of the saturating light intensity) for a particular organism can be thought of as “low” light intensities.

Alternatively or additionally, in certain embodiments, the plurality of strains can be grown in a low CO₂ environment. When the strains are cultured in liquid (or on solid) media, the concentration of CO₂ in the medium can be about 10 μM or less, for example about 1 μM or less, about 100 nM or less, about 10 nM or less, or about 1 nM or less. In certain embodiments, when CO₂ is present in a (solid/liquid) medium via diffusion from a gas, a low CO₂ environment can be provided by contacting the (solid/liquid) medium with a CO₂-poor gas (e.g., less than about 4 ppm w/v, less than 1 ppm w/v, less than about 0.4 ppm w/v, or less than about 0.04 ppm w/v). Examples of such embodiments can include, but are not limited to, bubbling a CO₂-poor gas through a liquid phase culture, which can also simultaneously serve to agitate the culture, and exposing the head-space above a solid phase culture to a CO₂-poor gas—in both cases, the CO₂-poor gas can provide the source of CO₂, which then diffuses through the solid/liquid media to offer the low CO₂ environment to the strains, via said media.

In other embodiments, the plurality of strains can be grown in an environment with higher CO₂. When the strains are cultured in liquid (or on solid) media, the concentration of CO₂ in the higher CO₂ environment can be at least about 20 μM, at least about 50 μM, at least about 100 μM, at least about 500 μM, or at least 1 mM. In certain embodiments, when CO₂ is present in a (solid/liquid) medium via diffusion from a gas, a higher CO₂ environment can be provided by contacting the (solid/liquid) medium with a gas having a CO₂ concentration of at least 5 ppm w/v, for example at least 10 ppm w/v, at least 50 ppm w/v, at least 100 ppm w/v, at least 500 ppm w/v, or at least 1000 ppm w/v.

In various embodiments, the plurality of strains can be grown in medium containing little to no nitrogen. For example, the strains can be grown in a medium containing about 10% to about 90% of a growth saturating concentration of nitrogen, e.g., about 10% to about 80%, about 10% to about 60%, about 10% to about 50%, about 10% to about 40%, about 20% to about 90%, about 20% to about 80%, about 20% to about 60%, about 20% to about 50%, about 20% to about 40%, about 30% to about 90%, about 30% to about 80%, about 30% to about 70%, about 30% to about 60%, about 30% to about 50%, about 40% to about 90%, about 40% to about 80%, about 40% to about 70%, about 40% to about 60%, about 50% to about 90%, about 50% to about 80%, or about 50% to about 70%. As used herein, the “growth saturating amount” of nitrogen is any amount at which the nitrogen concentration is high enough as not to be a rate limiting factor for growth and division. Therefore, the “growth saturating” amount of nitrogen can vary from organism to organism and also according to other environmental conditions such as tempterature, salinity, illumination, etc. In embodiments in which NH₄Cl is added to the medium to supply nitrogen, the concentration of NH₄Cl in the culture medium can be equal to or greater than ˜0 mM, e.g., at least 1 mM, at least 2 mM, at least 3 mM, at least 4 mM, at least 5 mM, at least 6 mM, or even at least ˜7.1 mM. Similarly, the total molar concentration of nitrogen in the medium can be equal to or greater than ˜0 mM, e.g., at least 1 mM, at least 2 mM, at least 3 mM, at least 4 mM, at least 5 mM, at least 6 mM, or even at least ˜7.1 mM.

The temperature of the culture medium is not particularly limiting, and can be, for example, ˜10° C., ˜15° C., ˜20° C., ˜25° C., ˜30° C., ˜35° C., or even ˜40° C. For example, the culture can be grown in a medium that changes during the course of a day from (e.g.) ˜10° C. to ˜35° C., or from (e.g.) ˜21° C. to ˜30° C.

### Methods of Strain Selection

The methods described herein can select strains of algal cells with enhanced photosynthetic efficiency. Previously, methods of selecting for enhanced photosynthetic efficiency were limited to methods of artificial selection, where an investigator would measure oxygen elaboration, biomass accumulation, or some other such efficiency-related trait of each of the strains under investigation. The investigator would then have to advance the strains with the most advantageous efficiency traits to the next round of selection or to industrial application. These methods, however, lacked the power of natural selection. The present disclosure solves this problem by describing methods that can impose a fitness cost on strains that do not use photosynthetically active radiation (“PAR”) more efficiently than their neighbors.

To take advantage of these directed evolution techniques, an algal strain of interest can be mutagenized to generate an assortment of mutants. In certain embodiments, the parental strain to be mutagenized is a wild-type strain. In other embodiments, the parental strain to be mutagenized is a strain that has already been mutated and/or modified, e.g., a strain that has been isolated from one or more rounds of selection to enhance photosynthetic efficiency. Following the mutagenesis, two or more of these mutants can then be cultured together in a turbidostat. In certain embodiments, the wild-type parent can also be co-cultured in the culture with the mutants under selection. In at least one round of selection, the cells can be cultured under high light (e.g., at least 500, at least 1000, at least 2000, at least 3000, or at least 4000 μEm⁻¹s⁻²). Because the turbidostat continuously dilutes the culture as the optical density increases, the culture is progressively enriched with cells that are able to grow and divide rapidly and efficiently under high light conditions. In certain embodiments, the “high light” conditions are conditions that are photoinhibitory to the wild-type algal strain from which the mutants are derived.

The rate at which selection occurs can depend on the fitness traits of the strains being selected and the strenuousness of the selection conditions. For example, in appropriate nutrient-rich conditions, Tetraselmis algae in logarithmic phase divide every 20 hours (see, Gopinathan (1986) Indian J. Fisheries 33(4):450-56). Therefore, if two competing strains begin at equal proportion in a co-culture, and one strain of Tetraselmis cells (strain A) has a 5% fitness advantage over another strain (strain B) in the context of a particular trajectory, it requires only 17.5 days under the selection criteria before A can be twice as prevalent in the culture as B. Of course, the period of time necessary to achieve this degree of enrichment of the more fit strain can be less if the fitness advantage is starker (such as 10%, 15%, 25%, etc.), and the time can be longer if the fitness advantage is smaller (such as 4%, 1%, 0.5%, etc.). The period of time necessary to enrich for the more fit strain can also vary according to the proportion of cells from each strain at the start of the selection conditions. That is to say, if the strains do not start in equal proportion, it can require more or less time for the more fit strain to emerge as dominant.

Without being bound by any particular mechanism, the methods described herein are unique in that they can select for both reduced antenna size and resistance to photoinhibition simultaneously. Either reduced antenna size or enhanced resistance to photoinhibition would, by itself, help to increase biomass productivity. Reducing the antenna size takes pigment out of the light harvesting complexes, thus allowing more photons to penetrate past the outermost layers of cells, allowing more efficient use of photon energy, and also reach into the deeper parts of the culture. The mechanisms by which resistance to photoinhibition work are less well understood, but it is known that D1 reaction center proteins from different organisms have different responses to high light, so it could be that the methods described herein select for D1 protein mutations that are optimized for resisting photoinhibition. When strains of photosynthetic microbes are cultured in such a medium under conditions that are photoinhibitory for some cells, the cells for which these conditions are not photoinhibitory can progressively come to predominate in a mixed culture over less efficient strains.

In certain embodiments of these methods, strains can be cultured in a medium lacking a reduced carbon source, such that the only source of metabolic energy comes from photosynthesis. In certain embodiments, the natural selection forces of the methods described herein can be supplemented with artificial selection, e.g., the photosynthetic efficiency of individual strains isolated from co-culture can be tested and strains that show superior efficiency can be selected for subsequent rounds of co-culture and selection.

In certain embodiments, the strains in the co-culture can be subjected to multiple rounds of selection. For example, strains in co-culture can be subjected to 2, 3, 4, 5, 6, 7, 8, 9, 10, 20, 30, 40, 50, 60, 70, 80, 90, or even 100 or more rounds of selection. In this way, with each round of selection the culture can become more enriched for the strain with the desired trait. If desired, the selective pressure can be increased, gradually or acutely, over repeated rounds of selection, for example by increasing the length of time during which cells are exposed to a selective pressure. In certain embodiments, one particular strain, the “dominant” strain, can come to predominate numerically in the culture with time. In certain embodiments, it is desirable to isolate pure or substantially pure cultures of this dominant strain. Isolation techniques, such as limited dilution and soft-agar culture, are well known to those of skill in the art.

In certain embodiments, it can be desirable to measure cell responses to illumination conditions. Cells can respond to light stimulus in a variety of ways, including by responses that manifest as changes in parameters such as temperature, pH, carbon concentration, and/or oxygen concentration of the cell (physiological change) or its micro-environment (environmental change). Cells can also respond by changing their pigment compositions (including their chlorophyll composition), which can result in a change in cellular fluorescence. Measurements of these changes can be made in many ways. Where the cell emits a product in response to illumination conditions, such as a starch, a lipid, or a volatile organic compound, it may be possible to sample this effluence by removing samples of culture medium or off-gas from the culture. In other embodiments, it may be useful to inspect samples of the cells visually under a microscope, or via flow cytometry. In certain embodiments, when cells are examined by flow cytometry, it is possible to remove cells that do not display desired characteristics, so as to enrich the remaining growth culture for cells possessing desired traits.

In some embodiments, co-cultures include 2, 3, 4, 5, 6, 7, 8, 9, 10, 20, 30, 40, 50 60, 70, 80, 90, 100, 500, 1000, 2000, 3000, 5000, 10000, 50000, 1×10⁵, 5×10⁵, 1×10⁶, 5×10⁶, 5×10⁸, 1×10⁹, or more strains cultured together in a single turbidostat. The person of ordinary skill should appreciate that the number of strains can be larger or smaller, depending on the size of the genome and the degree to which it is necessary or desirable statistically to cover mutations throughout the whole genome. The turbidostat can be structured of dimensions and materials sufficient to ensure that no more than 40%, for example no more than 30%, no more than 25%, no more than 20%, no more than 15%, no more than 10%, no more than 5%, no more than 1%, no more than 0.5%, no more than 0.1%, no more than 0.05%, no more than 0.01%, no more than 0.005%, or even no more than 0.001% of light intensity is lost across the optical path length of the vessel. In other embodiments, the density of the culture is sufficient to result in a loss of more than 20%, for example more than 25%, more than 30%, more than 35%, or more than 40% of light intensity across the optical path length of the vessel.

The optical density for a body of water containing photosynthetic microorganisms can correspond to an absorption coefficient in Beer's Law. Optical density corresponds to an amount of absorption per centimeter (or another convenient length unit). Based on an optical density (or absorption coefficient) α and a path length L, Beer's law can be written as:

αL=−log₁₀(I_{L}/I₀)=εLc

where aL is the absorbance (Aλ ) of the culture at a particular wavelength, Io represents the intensity of light incident at the surface, and IL represents the intensity of light at distance L from the surface.

“Biomass productivity” in this context is dX/dt=μX, where this equation defines μ, the specific growth. It can be calculated, for example, according to the formula Ln(biomass (t₂)/biomass (t₁))/(t₂−t₁), where t₂ is a time point arbitrarily later than t₁. When the ash free dry weight of a dense culture is measured in g/L, one can multiply by the volume (liters) of culture per square meter of illuminated surface to get grams of ash free dry weight/m²/day as the productivity of the simulated dense culture.

The turbidostat for use in the methods described herein maintains the optical density of a culture at or below a certain limit. Although the optical density can be measured at any wavelength, in certain embodiments optical density is measured at 730 nm, which is believed to correlate with cell concentration. The optical density can be maintained at any density measure, e.g., at about 0.6 or less, about 0.5 or less, about 0.3 or less, about 0.2 or less, about 0.1 or less, or about 0.01 or less. For an efficient selection process based on high light, the optical density may additionally or alternatively be maintained, for example at the lowest value for which the turbidostat operates stably.

### Growth Conditions

In certain embodiments, the turbidostat in which the cultures are grown can also allow for control of one or more additional variables related to photosynthetic microbial growth, such as but not limited to temperature, salinity, pH, carbon dioxide concentration, oxygen concentration, and/or nitrogen concentration.

One set of considerations for the turbidostat can be the size and shape, which should be selected so that the turbidostat can hold a sufficient volume of photosynthetic microorganisms to allow for desired characterization or testing of the photosynthetic microorganisms. Additionally, the turbidostat can have a shape that reduces or mitigates light intensity attenuation for the photosynthetic microorganisms inside.

The turbidostat volume can be selected to hold a desired sample volume of water or growth media. An optical density of about 0.1 can correspond to a biomass density of about 0.03 g/L to about 0.05 g/L for some types of photosynthetic microorganisms. One way of characterizing the growth rate of photosynthetic microorganisms can be to measure the ash free dry weight of the photosynthetic microorganisms. For this type of measurement to be reproducible, an average sample should contain at least a few milligrams of photosynthetic microorganisms, such as at least about 10 mg of photosynthetic microorganisms. Thus, for measuring photosynthetic microorganisms growth for an optical density of about 0.1, it is generally beneficial to have a sample volume (and therefore a corresponding turbidostat volume) of at least about a liter.

Alternatively, in some methods provided herein, the photosynthetic microorganisms in the turbidostat can be grown to a higher density, for example to an optical density (e.g., at ˜730 nm) of 0.5 or greater, for example 1.0 or greater or 1.5 or greater. In these methods, significantly smaller algal test culture volumes can be used, such as, for example, volumes of 500 mL or less, 250 mL or less, 100 mL or less, or 50 mL or less.

To allow in light, the turbidostat should be constructed of a material that is transparent or substantially transparent to the incident light used for illuminating photosynthetic microorganisms in the vessel. Suitable materials for the container can include various types of clear glass or plastic. Clear polycarbonate plastic is one useful structural material, as polycarbonate facilitates sterilization of the growth vessel prior to the beginning of a test. Although ultraviolet light is typically not involved in photosynthesis reactions, it may be photoinhibitory to certain photosynthetic mechanisms. So the turbidostat may allow penetration of UV light, or not, for example depending upon whether resistance to UV light photoinhibition is part of the selection experiment. As an alternative, any attenuation of light by the structural material for the growth vessel can be accounted for by using a correspondingly stronger illumination source, so that the light intensity incident on the surface of the water can approximately match the desired model intensity from an illumination profile.

The turbidostat can include other features to allow for control of the reaction conditions in the vessel. For example, turbidostat temperature can typically also be controlled, as microbial growth rates are often strongly influenced by temperature. Methods and devices for controlling turbidostat temperature are known in the art.

Another factor that can be controlled in the turbidostat is the pH of the water. In many embodiments, CO₂ can be the primary acidic component in the water, and therefore the pH can be controlled by controlling the CO₂ content. CO₂ can be introduced into the culture via an inlet that allows for bubbling CO₂. Alternatively, an aeration port can be used to introduce CO₂. A flow meter or another convenient device can be used to control the input flow rate of CO₂. It may be desirable to hold the CO₂ concentration at a relatively constant value. Both pH and CO₂ concentration can be measured with devices and techniques known in the art.

Still another factor that can be controlled in the turbidostat is the oxygen content. Photosynthetic microorganisms can produce molecular oxygen as a by-product of photosynthesis. An inlet can be included in the growth vessel to allow for addition of oxygen, and/or an aeration port can be included to allow for removal of oxygen as desired.

Because nitrogen content can have an important influence on biomass production, it can be important to control nitrogen content of the medium. Although those skilled in the art should be familiar with the many well-known methods for controlling nitrogen content of a culture, perhaps the simplest and most efficient is simply to add nitrogen salts when more high-nitrogen conditions are desired, and to dilute nitrogen rich cultures with nitrogen-free, defined medium when lower nitrogen conditions are desired. The person of ordinary skill should also appreciate that there are many other nutrients/supplements (e.g., phosphorus, iron, sulfur, trace metals, salts, acids/bases, or the like, or combinations thereof) whose concentration in the medium can additionally or alternatively be controlled/restricted. Just as with nitrogen, when these other nutrients/supplements are restricted, the restriction can provide additional information and/or selection criteria.

In addition to inputs to the growth vessel, the turbidostat can also include features to allow for movement of water or growth media within the growth vessel. In examples where the sample volume is small, for example, about 1 liter or less, bubbling of air or CO₂ through the culture, for example, by insertion of a tube into the culture, can provide adequate culture mixing. An alternative or additional option can be to include a mechanical agitator to increase mixing. In further embodiments, a sparging mechanism can be used to provide movement or agitation of the water/growth medium in the growth vessel. As an example, a sparging mechanism could be used for introduction of CO₂ into the growth vessel.

### Growth Media

Photosynthetic microorganisms for use in the methods described herein can be grown in any suitable culture medium, including media well known to those of skill in the art. Solid and liquid growth media are generally available from a wide variety of sources, as are instructions for the preparation of particular media suitable for a wide variety of strains of microorganisms. For example, various fresh water and salt water media can include those described in Barsanti, L. and Gualtieri, P. (2005) Algae: Anatomy, Biochemistry, and Biotechnology, CRC Press, Taylor & Francis Group, Boca Raton, Fla., USA, which is incorporated herein by reference for media and methods for culturing photosynthetic microorganisms, or in the examples of WO 2008/151149, which is herein incorporated by reference. One useful culture medium is BG-11, the ingredients of which are given in Table 1. Another useful culture medium is Guillard's f/2 culture medium, supplemented with nitrogen, phosphorus, and iron.

TABLE 1
BG-11 Medium (ATTC)

| NaNO₃ | 1.5 | g |
|---|---|---|
| K₂HPO₄ | 0.04 | g |
| MgSO₄ * 7H₂O | 0.075 | g |
| CaCl₂ * 2H₂O | 0.036 | g |
| Citric acid | 6.0 | mg |
| Ferric ammonium citrate | 6.0 | mg |
| EDTA | 1.0 | mg |
| Na₂CO₃ | 0.02 | g |
| Trace Metal Mix A5* | 1.0 | ml |
| Agar (if needed) | (up to) 10.0 g | |
| Distilled water | 1.0 | L |

*Trace Metal Mix A5 Composition

| | |
|---|---|
| H₃BO₃ | 2.86 g |
| MnCl₂ * 4H₂O | 1.81 g |
| ZnSO₄ * 7H₂O | 0.22 g |
| Na₂MoO₄ * 2H₂O | 0.39 g |
| CuSO₄ * 5H₂O | 0.080 g |
| Co(NO₃)₂ * 6H₂O | 49.4 mg |
| Distilled water | to 1.0 L |

### Trait Measurement

After a desired period of growth in the turbidostat under selective pressure, one or more tests can be performed on the photosynthetic microorganisms to identify samples with traits correlating with or indicative of improved photosynthetic efficiency. In certain embodiments it can be useful to calculate the specific growth rate of one or more strains in the culture over a period of time. In certain embodiments, it may be useful to measure the ash free dry weight of a sample. Exemplary techniques for measuring ash-free dry weight are described below.

Another type of measurement can be a measurement of the total organic carbon in a sample. Many variations for total organic carbon analysis are available. Methods for total organic carbon analysis typically involve an initial acidification of a sample to drive dissolved CO₂ out of the sample. The sample can then be combusted or oxidized by various methods, and the CO₂ evolved from combustion/oxidation can be measured as an indication of carbon content. The evolved carbon can be measured by measuring a conductivity of the sample before and after evolution of CO₂, or by non-dispersive infrared analysis. For example, total organic carbon for an algal sample can be analyzed using a SHIMADZU TOC-VCSH Analyzer, which can efficiently oxidize organic compounds, or by various commercially available CHN analyzers.

Still another type of characterization can be lipid productivity. For example, the total amount of lipids present in a sample can be measured by gas chromatographic fatty acid methyl ester (FAME) analysis. Lipid productivity can be useful for measuring the capability of a photosynthetic microbial sample for generating the lipid products which can eventually be converted into a diesel fuel or other valuable product. For methylation of free fatty acids and transmethylation of lipids the AOCS method Ce 1j-07 is used with some modifications, followed by alkali hydrolysis and methylation.

To determine the fatty acid ester content by FAME analysis, an algal culture sample (˜2 mL) can be lyophilized to dryness followed by alkali hydrolyses with ˜700 μL of ˜0.5 M KOH in methanol/tetrahydrofuran (˜2.5:1) mix. Glass beads can be added to the tubes, which can then be vortexed and then heated at ˜80° C. for ˜5 mins. The tubes can be allowed to cool ˜5 mins at room temperature (˜20-25° C.), before methylation with ˜500 μL of ˜10% BF₃ at ˜80° C. for ˜30 mins. Vials can then be allowed to cool ˜5 mins before extraction with ˜2 mL of heptane and ˜500 μL of ˜5 M NaCl. After vortexing, samples can be centrifuged for ˜1 min at ˜2000 rpm to separate phases. About 0.9 μl of the hexane extract can be injected into an Agilent 7890A gas chromatography system at a flow rate of ˜0.5 mL/min hydrogen at ˜100° C. for about 1 min, followed by a relatively fast temperature gradient to ˜230° C. for ˜1.7 mins. A DB-FFAP capillary column (J&W Scientific) can be used, ˜10 m long with ˜0.10 mm diameter and ˜0.10 μm film thickness. The inlet can be held at ˜250° C., and the FID detector at ˜260° C.

Peaks can be identified based on external standards. Absolute areas for both analytes and the internal standards can be obtained and the amount of FAME calculated for each sample. The efficiency of derivatization of triacylglycerides can be determined by computing the ratio between FAME originating from a triacylglyceride internal standard (e.g., C13:0) and FAME originating from a FAME internal standard (e.g., C23:0). The efficiency of derivatization of fatty acids can be determined by computing the ratio between FAME originating from an internal standard free fatty acid (e.g., C11:0) and FAME originating from an internal standard FAME (e.g., C23:0) (The ratios should be close to 1).

To determine the total organic carbon (TOC) content of algal cells, samples of cell cultures can be centrifuged to remove media and resuspended in water. Cell samples (three per measurement) can be injected into a SHIMADZU TOC-Vcsj Analyzer (or other commercial analyzer) for determination of Total Carbon, Total Inorganic Carbon, and, optionally, Total Nitrogen. The combustion furnace can be set to ˜720° C., and TOC can be determined by subtracting TIC from TC. The calibration range can be from ˜2 ppm to ˜200 ppm. The correlation coefficient requirement is preferably r²>0.99.

In addition, cultures can be tested for photosynthetic properties, including, for example, Fᵥ/Fₘ, oxygen evolution, and non-photochemical quenching. The cultures can be used to assay for or perform chemical analysis to detect metabolites, pigments, particular lipids, cofactors, or enzymes. The cultures can also be tested for expression of particular genes or production of proteins, for example using PCR, nucleic acid hybridization, antibody detection, or other techniques.

### Types of Photosynthetic Microorganisms

A photosynthetic microorganism for use in the methods described herein can include any isolate of a photosynthetic microbial species or subspecies, and includes mutants and genetically engineered strains. Photosynthetic microorganisms considered herein can include, but are not limited to, unicellular and multicellular photosynthetic microorganisms. Examples of such photosynthetic microorganisms can include a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof. In one embodiment, photosynthetic microorganisms can be of the classes Chlorophyceae and/or Haptophyta, Bacillariophyceae, Eustigmatophyceae, Trebouxiophyceae, or Prasinophyceae. Specific species can include, but are not limited to, Neochloris oleoabundans, Scenedesmus dimorphus, Euglena gracilis, Phaeodactylum tricornutum, Pleurochrysis carterae, Prymnesium parvum, Tetraselmis chui, Nannochloropsis gaditana, Dunaliella salina, Dunaliella tertiolecta, Chlorella vulgaris, Chlorella variabilis, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Picochlorum, Platymonas, Pleurochrysis, Pleurococcus, Prototheca, Pseudochlorella, Pseudoneochloris, Pyramimonas, Pyrobotrys, Scenedesmus, Schizochlamydella, Skeletonema, Spyrogyra, Stichococcus, Tetrachorella, Tetraselmis, Thalassiosira, Viridiella, or Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Tolypothrix, Trichodesmium, Tychonema, and Xenococcus species.

Oils or lipids are typically contained in photosynthetic microorganisms in the form of membrane components, storage products, and metabolites. Certain algal strains, particularly microalgae such as diatoms, certain chlorophyte species, and cyanobacteria, contain proportionally high levels of lipids. Algal sources for the algae oils can contain varying amounts, e.g., from 2 wt % to 40 wt % of lipids, based on total weight of the biomass itself. The systems and methods described herein can be used to evaluate production of lipids by algae strains. In certain strains that show enhanced specific growth rate under high light, the increase can be at least partially due to an increase in carbon fixation rates and/or can be at least partially due to a decrease in photoinhibition. In certain embodiments, a decrease in photoinhiition can be attributed to a reduction in active photosystems.

### Mutagens

The choice of mutagen for use in mutagenizing photosynthetic microorganisms is not particularly limiting. Mutagens such as ionizing radiation (e.g., ultraviolet radiation), chemical intercalating agents (e.g., ethidium bromide, proflavine, etc.), reactive oxygen species (e.g., hydrogen peroxide, nitrous oxide, etc.), deaminating agents (e.g., nitrous acid), polycyclic aromatic hydrocarbon (PAH), alkylating agents (e.g., ethylnitrosourea, nitrosoguanidine, methyl methanesulfonate, etc.), aromatic amines and amides (e.g., 2-acetylaminofluorene), vegetable alkaloids, psoralen, and benzene are well known to the art. Transposons and retrotransposons are also useful for generating large numbers of mutants, including libraries of mutants for screening and directed evolution applications. In certain embodiments, the mutagenization process proceeds so briefly that only a subset of host genes are mutated. In certain embodiments—particularly embodiments that use transposable elements as the mutagen—it is possible to do targeted mutagenesis to make it more likely that only certain, pre-ordained genes are mutated. In certain embodiments, the mutagenization process can proceed for an intensity and duration sufficient to ensure that all genes in the target organism can acquire at least one mutation. In certain embodiments, the mutagenization process can proceed for an intensity and duration sufficient to ensure that every base pair position in the target organism's genome can be mutated in at least one resulting organism.

### Isolated Strains and Pure Cultures

As desired throughout the course of the selection process, the operator can isolate individual strains in pure culture from out of the mixed culture in the turbidostat. Techniques for isolating and purifying microalgal cultures—such as limiting dilution—are known in the art. In certain embodiments, the isolates can be deposited in a depository institution, such as the American Type Culture Collection. In certain embodiments, this deposit can be made under the Budapest Treaty.

In certain embodiments, the isolates can produce smaller light-harvesting antenna than their corresponding parental strains. In certain embodiments, the isolates can possess proteins in their photosynthetic reaction center complexes (e.g., the D1 protein) that can be more resistant to photodamage than are the corresponding proteins in the relevant parental strains. In certain isolates, all of the photosynthetic efficiency enhancement can be the result of a single mutation, while in other isolates the overall efficiency enhancement can be the additive (or even super-additive) effect of more than one mutation. In certain embodiments, the acquired mutations can result in a photosynthetic efficiency enhancement of at least 5%, for example at least 10%, at least 15%, at least 25%, at least 50%, at least 75%, at least a 2-fold increase, at least a 5-fold increase, or even at least a 10-fold increase relative to the parental strain.

In certain embodiments, the resulting isolate may not become photoinhibited by PAR that is at least 5% more intense than the PAR that would photoinhibit the corresponding parental strain, for example at least 10%, at least 15%, at least 25%, at least 50%, at least 75%, at least 2-fold more, at least 5-fold more, or even at least 10-fold more PAR. Additionally or alternatively, the resulting isolate may display an enhanced photosynthetic rate relative to the parental strain, for example at least 10%, at least 15%, at least 25%, at least 50%, at least 60%, at least 65%, at least 70%, at least 75%, at least 2-fold more, at least 5-fold more, or even at least 10-fold faster photosynthesis. Additionally or alternatively, the isolate may demonstrate an enhanced rate of carbon fixation relative to the parental strain, for example at least 10%, at least 15%, at least 25%, at least 50%, at least 60%, at least 65%, at least 70%, at least 75%, at least 2-fold more, at least 5-fold more, or even at least 10-fold faster carbon fixation.

These isolated strains can have a number of uses. Because they, by definition, have already been selected based—at least in part—on their enhanced photosynthesis efficiency, these isolates can be used as parental strains for further mutagenesis and optimization, e.g., further reduction of the light-harvesting antennae. In addition, they can also be useful in industrial biomass and biosynthesis operations, where their enhanced photosynthesis efficiency should result in more product generation for a given quantum of PAR.

Having once isolated a strain, in certain embodiments, it may be desirable to map the mutations that have arisen in the enhanced-efficiency strains. For example, in certain embodiments, the isolate's genome can be sequenced (e.g., using next generation sequencing). Where the mutation was introduced with a transposable element, the site of the mutation can be mapped with PCR based techniques. In certain embodiments, once the site of the mutation is known in the isolate, this same mutation can be introduced into a wild-type cell to test whether the mutation recapitulates in a fresh cell the high-light tolerance phenotype observed in the isolate.

### Genetic Engineering

Additionally or alternatively, in certain embodiments, an isolated strain obtained by the methods described above can be the subject of genetic engineering. In certain embodiments, the isolate can be manipulated to include other mutations identified by other iterations of the selection methods described above. In certain embodiments, the isolate can be modified to include other mutations known to affect photoinhibition, photosynthesis rate, and/or the rate of carbon fixation. In certain embodiments, the isolate can be modified to include additional mutations to reduce antenna size. In certain embodiments, the isolate can be modified to include one or more (sets of) mutations that can affect any of a variety of relevant parameters, including but not limited to salt tolerance, drug tolerance, ability to produce one or more enzymes necessary to a given reaction or set of reactions (e.g., biosynthetic reactions), ability to degrade or utilize a given chemical precursor, ability to produce an antibiotic, etc.

### Breeding

Certain photosynethetic microorganisms are capable of both sexual and asexual reproduction. For example, C. reinhardtii can be mated to produce hybrid algal strains. Jiang & Stern (2009) JoVE 30:1274, doi:10.3791/1274. In certain embodiments, isolates produced by the methods described above can be mated with other algal strains to produce a hybrid strain incorporating advantageous traits from both ancestral strain lineages. For example, in one exemplary embodiment, one can cross a strain selected for rapid growth under high light and a strain selected for rapid growth under low light. If the mutation giving rise to the high-light adaptation and the mutation giving rise to the low-light adaptation are independent of each other, then it should be possible to breed a strain that possesses both mutations, thus rendering the resulting hybrid well-adapted to both high and low light conditions.

By way of nonlimiting example, repeated iterations of the mutation/selection methods described above can produce a variety of different mating types of the same algal species, such that one can obtain a mutant with high specific growth rate, but little to no reduction in antenna size, and another mutant with pronounced reduction in antenna size, but no appreciable improvement in specific growth rate. In such circumstances, one could could breed these two strains to obtain a hybrid with both traits. In certain embodiments, one or both of those two mutant mating strains could be genetically engineered, as described above, before the hybridization. In certain embodiments, the resulting hybrid strain could be genetically engineered as described above. In certain embodiments, the resulting hybrid strain could be subject to additional rounds of mutagenesis and/or selection, as desired.

Breeding techniques and conditions are known to those of ordinary skill. Known techniques for cross-breeding may be adjusted and optimized as necessary to the relevant (algal) strain selected according to the methods described above.

Methods of Generating Biomass

Methods of generating biomass are also disclosed herein. Once a strain has been isolated following the selection methods described above, this strain can be cultivated for production of biomass or particular biomolecules, such as lipids, proteins, and/or carbohydrates. Additionally or alternatively, once an isolate has been sequenced and the mutation responsible for photosynthesis efficiency enhancement has been identified, the mutation can be introduced into another cell, and cultures of this newly modified cell can be cultured for biomass accumulation or for the production of a particular biomolecule or set of biomolecules.

Although the methods of biomass and biomolecule production described herein do not require that the photosynthetic microorganisms be cultured in a turbidostat, in other respects the description above regarding growth conditions are largely similar for the methods of biomass accumulation as for the methods of strain selection. Illumination, salinity, pH, temperature, and nitrogen, oxygen, and carbon concentrations can be adjusted as necessary to optimize accumulation of the biomass and/or biomolecule of interest. Culturing refers to the intentional fostering of growth (e.g., increases in cell size, cellular contents, and/or cellular activity, e.g., biomolecule synthesis) and/or propagation (e.g., increases in cell numbers via mitosis) of one or more cells by use of selected and/or controlled conditions. The combination of both growth and propagation may be termed proliferation. Non-limiting examples of selected and/or controlled conditions can include the use of a defined medium (with known characteristics such as pH, ionic strength, and/or carbon source), specified temperature, oxygen tension, carbon dioxide levels, growth in a bioreactor, mixing of the culture, or the like, or combinations thereof. In some embodiments, the microorganism can be grown heterotrophically or mixotrophically, using both light and a reduced carbon source.

In certain embodiments, the microorganism can be cultured phototrophically. When growing or propagating phototrophically, the microorganism can advantageously use light as an energy source. An inorganic carbon source, such as CO₂ or bicarbonate, can be used for synthesis of biomolecules by the microorganism. “Inorganic carbon”, as used herein, includes carbon-containing compounds or molecules that cannot be used as a sustainable energy source by an organism. Typically “inorganic carbon” can be in the form of CO₂ (carbon dioxide), carbonic acid, bicarbonate salts, carbonate salts, hydrogen carbonate salts, or the like, or combinations thereof, which cannot be further oxidized for sustainable energy nor used as a source of reducing power by organisms. If an organic carbon molecule or compound is provided in the culture medium of a microorganism grown phototrophically, it generally cannot be taken up and/or metabolized by the cell for energy and/or typically is not present in an amount sufficient to provide sustainable energy for the growth of the cell culture.

The photosynthetic microorganism can be cultured as an actively mixed culture, for example in a pond or photobioreactor. For example, the photosynthetic microorganism can be cultured in a pond having a depth of at least 3 cm, at least 5 cm, or at least 10 cm, or a photobioreactor having a light path of at least 3 cm, at least 5 cm, or at least 10 cm. As used herein “pond” means any open body of water, whether naturally-occurring or man-made, including ponds, canals, trenches, lagoons, channels, or raceways. The pond or bioreactor can include at least one active mixing device, such as a paddlewheel, pump, propeller, fluid injection system, sparger, or any combination thereof, optionally in combination with at least one passive mixing device. Further additionally or alternately, the photosynthetic microorganism can be cultured in a volume of at least 20 liters of culture medium.

In some embodiments, the amount of biomass or of a biomolecule produced by the culture can be at least 10%, for example at least 15%, at least 20%, or at least 25%, greater than the amount of a biomolecule produced by an identical culture of a control microorganism that has not been produced by the selection methods described above. Additionally or alternately, the photosynthetic microorganism can be cultured phototrophically and/or under intermittent light conditions, e.g., in an actively mixed culture, optionally under natural light.

In further aspects, the microorganisms can be cultured in a suitable culture medium, which in some examples can be a culture medium that does not include a substantial amount of a reduced carbon source, such that the cells are cultured photoautotrophically. Additionally, the culture medium can include inorganic carbon as substantially the sole source of carbon for production of the biomolecule.

A source of inorganic carbon (such as, but not limited to, CO₂, bicarbonate, carbonate salts, and the like), including, but not limited to, air, CO₂-enriched air, flue gas, or the like, or combinations thereof, can be supplied to the culture. When supplying flue gas and/or other sources of inorganic that may contain CO in addition to CO₂, it may be necessary to pre-treat such sources such that the CO₂ level introduced into the (photo)bioreactor do not constitute a dangerous and/or lethal dose vis-à-vis the growth and/or survival of the microorganisms.

Culturing of photosynthetic microorganisms can be performed under various conditions, such as under a light/dark cycle, and/or under natural light. In some embodiments, light/dark cycle refers to providing and removing (e.g., switching on and off) the light over a predetermined period, for example, a light dark cycle can be 12 hours of light followed by 12 hours of darkness or 14 hours of light followed by 10 hours of darkness. Alternatively or in addition, the light/dark cycle can be a natural light/dark cycle based on day-length, where the sun is the light source. Natural light can optionally be supplemented by artificial light. In some culture systems, the light period of a culture grown under natural light can be extended by the inclusion of one or more artificial light sources. When the cultures are grown under artificial light, the light can be of any amount of photosynthetically active radiation, for example at least 50, at least 100, at least 250, at least 500, at least 750, at least 1000, at least 1500, at least 2000, at least 3000, at least 4000, at least 5000, at least 7500, or even at least 10000 μEm⁻²s⁻¹.

The depth of a pond or light path of a photobioreactor also plays a factor in the amount of mixing that is needed to achieve a desired level of turbulence. For example, the depth of the growth pond can have a substantial impact on the Reynolds number for the pond. As an illustrative example, if the depth of the pond is reduced from 30 cm to 10 cm, the Reynolds number of such a pond can increase from about 1000 to about 3000.

Biomass of the microorganism culture can be recovered by harvesting the microorganism from the medium, for example, by filtering, settling, flotation, centrifugation, or combinations thereof. In biomass production embodiments, the amount of the biomass produced and/or recovered by the method described herein, measured as ash free dry weight (AFDW) can advantageously be at least about 0.05 g per liter of culture, for example at least about 0.1 g, at least about 0.2 g, at least about 0.3 g, at least about 0.4 g, at least about 0.5 g, at least about 0.6 g, at least about 0.7 g per liter of culture, at least about 1 g per liter of culture, at least about 1.5 g per liter of culture, at least about 2 g per liter of culture, at least about 2.5 g per liter of culture, or at least about 5 g per liter of culture. Although many times the goal can be to produce and/or recover as much biomass as possible, in some instances the amount of the biomass produced and/or recovered by the method described herein, measured as ash free dry weigh (AFDW) can be limited to about 15 g or less per liter of culture, for example about 12 g or less per liter of culture, about 10 g or less per liter of culture, about 5 g or less per liter of culture, about 2 g or less per liter of culture, about 1 g or less per liter of culture, or about 0.5 g or less per liter of culture.

The dry weight (DW) and ash-free dry weight (AFDW) can be calculated according to the formulas:

$$DW ( g / l ) = \frac{( ovenweight ( g ) - filterweight ( g ) ) * 1000 ( ml / l )}{samplevolume ( ml )}$$

$$AFDW ( g / l ) = DW ( g / l ) - ( \frac{( furnaceweight ( g ) - filterweight ( g ) )}{samplevolume ( ml )} * 1000 ( ml / l ) )$$

Where “ovenweight” is the weight of the sample after drying in the oven, and “furnaceweight” is the weight of the same sample after combusting in the muffle furnace.

By way of representative procedure for measuring biomass, ˜25 to ˜35 mL of removed sample of each culture can be transferred to a filtration assembly that includes a sidearm flask fitted with a stopper, funnel, and screen for supporting a filter held with a clamp. A pre-weighed Whatman 47 mm GF/F glass microfiber filter can be positioned over the screen. The sample can be pipetted onto the surface of the filter, and a vacuum (about 5-10 psi) applied via the side arm of the flask. Once all the liquid passes through the filter, the sides of the funnel can be rinsed with ˜9-12 mL distilled water to bring down any cells that may have stuck to the side of the funnel. The rinsing step can be repeated, e.g. twice. Once the filtration is finished, the filter can be removed from the base with forceps. The filter can be placed in a pre-weighed aluminum weighing boat, and then the samples placed in a ˜105° C. drying oven until the weight is constant, e.g. at least four hours. The dried samples can then be placed in a dessicator to cool, and then the weigh boat plus filter weighed. Dry weight is calculated as:

$$DW ( g / l ) = \frac{( ovenweight ( g ) - vialweight ( g ) ) * 1000 ( ml / l )}{samplevolume ( ml )}$$

Samples can then be placed into a muffle furnace heated to ˜550° C. for ˜1 hour. The samples are then removed using tongs and transferred to the desiccator to cool to room temperature. When the samples are cool, they can be weighed using the same analytical balance used to weigh the dry samples.

Ash Free Dry Weight (in g/l) is calculated as follows:

$$AFDW ( g / l ) = DW ( g / l ) - ( \frac{( furnaceweight ( g ) - vialweight ( g ) )}{samplevolume ( ml )} * 1000 ( ml / l ) )$$

Biomass can be used in any of a number of ways, for example, it can be processed for use as a biofuel by generating syngas from the biomass, can be supplied to an anaerobic digester for production of one or more alcohols, or the biomass can be extracted to provide algal lipids, such as but not limited to monoglycerides, diglycerides, or triglycerides, fatty acid alkyl esters, fatty acids, and/or fatty acid derivatives.

The microorganisms according to some embodiments produce free fatty acids and fatty acid derivatives in an amount greater than the amount of free fatty acids and fatty acid derivatives produced by a control strain that has not been produced by the selection methods described above, but which is grown under identical conditions.

### Other Embodiments

Additionally or alternatively to any of the described methods and systems, one or more of the following embodiments are also contemplated:

Embodiment 1. A method for selecting a strain of photosynthetic microorganisms (e.g., eukaryotic algae or cyanobacteria) for increased biomass accumulation in photosynthetic culture based on specific growth rate, the method comprising: (a) exposing at least two different strains of photosynthetic microbes to at least 3000 μEm⁻²s⁻¹ (e.g., at least 3500 μEm⁻²s⁻¹, at least 4000 μEm⁻²s⁻¹, at least 4500 μEm⁻²s⁻¹, or at least 5000 μEm⁻²s⁻¹) photosynthetically active radiation for at least five hours, wherein the at least two different strains are co-cultured in a turbidostat, and wherein at least one strain is a mutant strain.

Embodiment 2. The method of embodiment 1, wherein exposure continues until the strain with the highest specific growth rate comprises at least 30% of the cells in the turbidostat (e.g., at least 50%, at least 75% of the cells, or even 100% of the cells in the turbidostat).

Embodiment 3. The method of Embodiment 1 or 2, further comprising step (b): isolating a culture of cells belonging to the strain with the highest specific growth rate.

Embodiment 4. The method of any one of the previous embodiments, further comprising exposing the at least two different strains to no more than 50 μEm⁻²s⁻¹ photosynthetically active radiation for at least five hours.

Embodiment 5. The method of any one of the previous embodiments, wherein the at least two different strains are cultured in medium containing approximately 10 μM CO₂ or less and/or containing only ˜10% to ˜90% of a growth saturating amoung of nitrogen.

Embodiment 6. The method of any one of the previous embodiments, further comprising a step (c) preliminary to step (a): mutagenizing a culture to generate a plurality of mutants, wherein the at least two different strains of step (a) include a mutant from step (c).

Embodiment 7. The method of any one of the previous embodiments, wherein the culture is treated with a means for mutagenizing (e.g., ionizing radiation, a chemical intercalating agent, and/or a transposable nucleotide element).

Embodiment 8. The method of Embodiment 7, wherein the concentration of means for mutagenizing and the duration of treatment are together sufficient to ensure that every gene in the microbial genome is mutated in at least one cell in the culture.

Embodiment 9. The method of any one of the previous embodiments, wherein the at least one mutant strain is selected from a library of mutant strains (e.g., a library of mutant algal strains).

Embodiment 10. The method of Embodiment 6, wherein after step (c), the mutagenized cells are screened for pigmentation (for instance, by fluorescence activated cell sorting) before the at least one mutant is added to the co-culture of step (a).

Embodiment 11. The method of Embodiment 10, wherein only those mutants that show at least a 30% reduction in chlorophyll content (e.g., at least a 50% reduction, at least a 60% reduction, at least a 70% reduction, or at least an 80% reduction) relative to wild type are added to the co-culture.

Embodiment 12. The method of Embodiment 8, further comprising: (d) exposing cells of the isolated culture to means for mutagenizing to create a new set of mutants; and (e) co-culturing at least two of these mutants in a turbidostat for another iteration of step (a).

Embodiment 13. The method of any one of Embodiments 3-12, further comprising sequencing the genome of the isolated culture, and optionally further comprising introducing a mutation identified during the sequencing into a wild-type cell.

Embodiment 14. The method of any one of Embodiments 3-13, further comprising: (f) mutagenizing a cell from the isolated culture to generate a plurality of mutants; and/or further comprising: (g) exposing at least two different strains to no more than 50 μEm⁻²s⁻¹ photosynthetically active radiation for at least five hours, wherein the at least two different strains are co-cultured in a turbidostat, and wherein at least one strain is a mutant strain generated in step (d).

Embodiment 15. The method of Embodiment 14, further comprising: (h) co-culturing at least two different strains in medium containing approximately 10 μM CO₂ or less, wherein the at least two different strains are co-cultured in a turbidostat, and wherein at least one strain is a mutant strain generated in step (d).

Embodiment 16. The method of Embodiment 14 or 15, further comprising: (i) co-culturing at least two different strains of algal cells in medium containing less than growth-saturating concentration of a nitrogen source, wherein the at least two different strains are co-cultured in a turbidostat, and wherein at least one strain is a mutant strain generated in step (d).

Embodiment 17. The method of Embodiment 15 or 16, wherein at least five hours of the co-culturing in either or both of steps (h) and (i) is conducted under illumination not less than 3000 μEm⁻²s⁻¹ photosynthetically active radiation.

Embodiment 18. An isolated culture of photosynthetic microbial cells produced by the method of any one of the previous embodiments, wherein the cells of the isolated culture exhibit enhanced photosynthetic efficiency relative to corresponding wild-type cells of the same species, optionally wherein the cell belongs to an algal genus selected from the group consisting of Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Elhpsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Picochlorum, Platymonas, Pleurochrysis, Pleurococcus, Prototheca, Pseudochlorella, Pseudoneochloris, Pyramimonas, Pyrobotrys, Scenedesmus, Schizochlamydella, Skeletonema, Spyrogyra, Stichococcus, Tetrachorella, Tetraselmis, Thalassiosira, Viridiella, and Volvox, optionally wherein the algal genus is a genus capable of sexual reproduction.

Embodiment 19. A method of hybridizing a trait into an algal strain, the method comprising mating at least one cell from the isolated culture of Embodiment 18 with another algal cell to produce a hybrid strain.

Embodiment 20. A method of producing biomass, the method comprising culturing the microbial cells of Embodiment 18 until ash-free dry weight of biomass has increased at least 10% from the ash-free dry weight of biomass at the start of culture.

### EXAMPLE

The following example is set forth so as to provide those of ordinary skill in the art with a complete disclosure and description of how to make and use the described methods and microbes, and are not intended to limit the scope of what the inventors regard as their invention nor are they intended to represent that the experiments below are all or the only experiments performed. Efforts have been made to ensure accuracy with respect to numbers used (e.g., amounts, temperature, etc.), but some experimental errors and deviations should be accounted for. Unless indicated otherwise, parts are parts by weight, molecular weight is weight average molecular weight, temperature is in degrees Centigrade, and pressure is at or near atmospheric.

### Mutagenesis and Selection

When photosynthetic algal strain A was acclimated to ≧400 μEm⁻²s⁻¹, it appeared to reach a maximum specific growth rate in diute culture of ˜0.125 h⁻¹. However, if the same strain were grown in sub saturating light, or if it had been in the dark overnight, its maximum specific growth rate following a shift to supersaturating PAR would likely be initially much higher than its acclimated maximum specific growth rate. For instance, when grown in dilute culture at 50 μEm⁻²s⁻¹ and transferred (also in dilute culture) to 950 μEm⁻²s⁻¹, the specific growth rate appeared to reach as high as ˜0.175 h⁻¹. Similarly, when photosynthetic algal strain A is cultured in the dark and then transferred to high light, the specific growth rate can be as high as ˜0.23 h⁻¹. This shows that the photosynthetic algal strain A can be capable of relatively high specific growth rates under relatively high PAR conditions.

However, these relatively high specific growth rates attained immediately after the light shift can start to decrease soon after the shift. After a few hours, the maximum specific growth rate can adjust to the light-acclimated rate of about ˜0.125 h⁻¹.

The productivity that photosynthetic microorganisms can attain in dense culture can be a function of the magnitude of its maximum specific growth rate. In large outdoor ponds, an algal cell may stay at supersaturating light for hours, so that even a cell which has been in the dark can have its specific growth rate substantially lowered as it stays in the upper levels of the pond.

To test whether this reversion to acclimated-specific growth rate could be avoided, photosynthetic algal strain A cells were exposed to the chemical mutagen methylmethanesulfate (MMS) for a sufficient time and at a dose sufficient to ensure that all genes in the photosynthetic algal strain A genome were mutated in at least one cell in the culture. The mutagenized cell suspension was cultured in a turbidostat at 3500 μEm⁻²s⁻¹. Initially, at least 90% of the cells died from the mutagenesis. After a few days of continuous culture in these conditions, a population of cells emerged in the turbidostat with specific growth rate of ˜0.25 h⁻¹. This population was comprised of both a mutant produced from the parental line and a strain selected from the laboratory environment, originating from cultivation of algal strains collected from a natural water source.

Isolates from this population were collected and characterized in cultures at pH 7.0±0.2, at a temperature of 29.0±0.3° C., and at a semicontinuous dilution rate of 50% volume/day. Details concerning the growth rates of the wild-type and mutantisolate are summarized in Table 2 below.

TABLE 2
Specific Growth Rate of photosynthetic algal strain A

| | PAR | Growth Rate | Remarks |
|---|---|---|---|
| Strain | (μEm⁻²s⁻¹) | (h⁻¹) | (Dilute Cultures) |
| WT | ~900 | 0.13 ± 0.03 | Culture acclimated to |
| | | | 900 μEm⁻²s⁻¹ |
| WT | ~2500 | 0.13 ± 0.03 | Culture acclimated to |
| | | | 2500 μEm⁻²s⁻¹ |
| WT | ~900 | 0.23 ± 0.03 | Culture grown at 50 μEm⁻²s⁻¹ |
| | | | and moved to 900 μEm⁻²s⁻¹ |
| | | | for 4 hrs. |
| Mutant | ~950 | 0.22 ± 0.02 | Culture acclimated to |
| | | | 950 μEm⁻²s⁻¹ |
| Mutant | ~3500 | 0.24 ± 0.03 | Culture acclimated to |
| | | | 3500 μEm⁻²s⁻¹ |

The Photosystem II antenna size in the mutant appeared to be reduced ˜50% relative to the parental photosynthetic algal strain A strain. The magnitude of this reduction was seen to vary according to environmental conditions, which can be attributed primarily to the change in the antenna size of the wild type under those environmental conditions. For example, the antenna length is reduced only ˜30% when the mutant photosynthetic algal strain A is grown in dense culture. These results are summarized in Table 3 below.

TABLE 3
Photosystem II Antenna Size in photosynthetic algal strain A

| | | An- | | Chlorophyll | |
|---|---|---|---|---|---|
| | | tenna | | content | |
| | PAR | size | Ratio | (% ash-free | Ratio |
| Strain | (μEm⁻²s⁻¹) | (nm²) | (mutant:WT) | dry wt.) | (mutant:WT) |
| WT | ~900 | ~1.6 | ~0.55 | ~2.5 | ~0.90 |
| Mutant | ~900 | ~0.87 | | ~2.2 | |
| WT | Solar day* | ~1.33 | ~0.67 | ~2.4 | ~1.2 |
| Mutant | Solar day* | ~0.89 | | ~2.9 | |

*“Solar day” data were collected in an outdoor facility in Baytown, TX, on 8 Sep. 2011.

The strain selected from the natural water source overtook the mutant with time. It generated ≧˜50% increase in biomass productivity over the original parental strain when grown in dense culture under continuous illumination at 900 μEm⁻²s⁻¹. These results are summarized in Table 4 below. As can be seen from these data, unlike the wild-type, the selected strain did not appear to show a decline in specific growth rate during continuous culture at high light. These data appear to demonstrate the efficacy of the selection methods described herein above, as well as the unexpectedly advantageous properties of the selected strain.

TABLE 4
Productivity of photosynthetic algal strain A

| | PAR | Grams of ash-free | Ratio |
|---|---|---|---|
| Strain | (μEm⁻²s⁻¹) | dry wt. per m² · day | (mutant:WT) |
| WT | ~900 | ~18.7 | ~2.0 |
| Selected | ~900 | ~36.6 | |
| WT | Solar day* | ~23.0 | ~1.3 |
| Selected | Solar day* | ~30.0 | |

Although the methods and microbes described herein have been described in terms of specific embodiments, they should not be understood as so limited. Suitable alterations/modifications for operation under specific conditions should be apparent to those skilled in the art. It is therefore intended that the following claims be interpreted as covering all such alterations/modifications as fall within the true spirit/scope of the present disclosure.

1. A method for selecting a strain of algal cells for biomass accumulation in photosynthetic culture based on specific growth rate, the method comprising: (a) exposing at least two different strains of algal cells to at least 3000 μEm⁻²s⁻¹ photosynthetically active radiation for at least five hours, wherein the at least two different strains are co-cultured in a turbidostat, and wherein at least one strain is a mutant strain.

2. The method of claim 1, wherein the at least two different algal strains are exposed to at least 5000 μEm⁻²s⁻¹ photosynthetically active radiation for at least five hours.

3. The method of claim 1, wherein exposure continues until the strain with the highest specific growth rate comprises at least 50% of the cells in the turbidostat.

4. The method of claim 3, wherein exposure continues until the strain with the highest specific growth rate comprises 100% of the cells in the turbidostat.

5. The method of claim 3, further comprising step (b): isolating a culture of cells belonging to the strain with the highest specific growth rate.

6. The method of claim 1, further comprising exposing the at least two different algal strains to no more than 50 μEm⁻²s⁻¹ photosynthetically active radiation for at least five hours.

7. The method of claim 1, wherein the at least two different algal strains are cultured in medium containing approximately 10 μmolar CO₂ or less.

8. The method of claim 1, wherein the at least two different algal strains are cultured in medium containing only approximately 10% to approximately 90% of a growth-saturating amount of nitrogen.

9. The method of claim 1, further comprising a step (c) preliminary to step (a):
mutagenizing an algal cell culture to generate a plurality of mutants, wherein the at least two different strains of step (a) include a mutant from step (c).

10. The method of claim 9, wherein the algal culture is treated with a means for mutagenizing selected from the group consisting of ionizing radiation, a chemical intercalating agent, and a transposable nucleotide element.

11. The method of claim 10, wherein the concentration of means for mutagenizing and the duration of treatment are together sufficient to ensure that every gene in the algal genome is mutated in at least one cell in the culture.

12. The method of claim 1, wherein the at least one mutant strain is selected from a library of mutant algal strains.

13. The method of claim 9, wherein after step (c), the mutagenized algal cells are screened for pigmentation before the at least one mutant is added to the co-culture of step (a).

14. The method of claim 13, wherein the mutagenized algal cells are screened by fluorescence activated cell sorting.

15. The method of claim 14, wherein only those mutants that show at least a 30% reduction in chlorophyll content relative to wild type are added to the co-culture.

16. The method of claim 15, wherein only those mutants that show at least a 80% reduction in chlorophyll content relative to wild type are added to the co-culture.

17. The method of claim 9, further comprising: (d) exposing cells of the isolated culture to means for mutagenizing to create a new set of mutants; and (e) co-culturing at least two of these mutants in a turbidostat for another iteration of step (a).

18. The method of claim 5, further comprising sequencing the genome of the isolated culture.

19. The method of claim 18, further comprising introducing a mutation identified during the sequencing into a wild-type cell.

20. The method of claim 18, further comprising: (f) mutagenizing a cell from the isolated culture to generate a plurality of mutants.

21. The method of claim 17, further comprising: (g) exposing at least two different strains of algal cells to no more than 50 μEm⁻²s⁻¹ photosynthetically active radiation for at least five hours,wherein the at least two different strains are co-cultured in a turbidostat, and wherein at least one strain is a mutant strain generated in step (d).

22. The method of claim 21, further comprising: (h) co-culturing at least two different strains of algal cells in medium containing approximately 10 μM CO₂ or less,wherein the at least two different strains are co-cultured in a turbidostat, and wherein at least one strain is a mutant strain generated in step (d).

23. The method of claim 22, wherein at least five hours of the co-culturing is conducted under illumination not less than 3000 μEm⁻²s⁻¹ photosynthetically active radiation.

24. The method of claim 17, further comprising: (i) co-culturing at least two different strains of algal cells in medium containing only approximately 10% to approximately 90% of a growth saturating amount of nitrogen,
wherein the at least two different strains are co-cultured in a turbidostat, and wherein at least one strain is a mutant strain generated in step (d).

25. The method of claim 24, wherein at least five hours of the co-culturing is conducted under illumination not less than 3000 μEm⁻²s⁻¹ photosynthetically active radiation.

26. An isolated culture of photosynthetic microbes produced by the method of claim 5, wherein the photosynthetic microbes of the isolated culture exhibit enhanced photosynthetic efficiency relative to corresponding wild-type cells of the same species.

27. The isolated culture of claim 26, wherein the photosynthetic microbe belongs to a species of eukaryotic algae capable of sexual reproduction.

28. A method of hybridizing a trait into an algal strain, the method comprising mating at least one cell from the isolated culture of claim 27 with another algal cell to produce a hybrid strain.

29. A method of producing biomass, the method comprising culturing the photosynthetic microbes of claim 26, said photosynthetic microbes comprising algal cells, until ash-free dry weight of biomass has increased at least 10% from the ash-free dry weight of biomass at the start of culture.
